(19) United States
(12) Patent Application Publication
(10) Pub. No.: US 2008/0176261 A1
(43) Pub. Date: Jul. 24, 2008

(54) Colorimetric Substrate and Methods for Detecting Poly(ADP-ribose) Polymerase Activity including PARP Enzymes PARP-1, VPARP, and Tankyrase-1

(75) Inventors: Amanda C. Nottbohm, Urbana, IL (US); Robin Shane Dothager, Gifford, IL (US); Karson S. Putt, Champaign, IL (US); Mirth T. Hoyt, Urbana, IL (US); Paul J. Hergenrother, Champaign, IL (US)

Correspondence Address: GREENLEE WINNER AND SULLIVAN P C, 4875 PEARL EAST CIRCLE, SUITE 200, BOULDER, CO, 80301, US

(73) Assignee: THE BOARD OF TRUSTEES OF THE UNIVERSITY OF ILLINOIS, Urbana, IL (US)

(21) Appl. No.: 11/624,528

(22) Filed: Jan. 18, 2007

(51) Int. Cl. C12Q 1/48; C07H 17/02

(52) U.S. Cl. 435/15; 536/17.4

(57) ABSTRACT

Disclosed are compositions and methods capable of facilitating the detection and measurement of poly(ADP-ribose)polymerases (PARP enzymes). PARP enzyme activity can be monitored using a novel calorimetric substrate, ADP-ribose-para-nitrophenol. The substrate can be synthesized from beta nicotinamide adenine dinucleotide (β-NAD⁺) and para-nitrophenol. In an embodiment, a continuous assay was developed to detect and kinetically monitor activity for PARP enzymes such as PARP-1, tankyrase-1 (PARP-5), and VPARP (PARP-4). The compositions and methods are particularly useful in the screening and identification of specific PARP inhibitors.

### CROSS-REFERENCES TO RELATED APPLICATIONS

Not Applicable.

### STATEMENT ON FEDERALLY SPONSORED RESEARCH OR DEVELOPMENT

Not Applicable.

### BACKGROUND OF THE INVENTION

Poly(ADP-ribose)polymerases (PARP) enzymes are proteins involved in many processes in the cell. These cellular processes mainly involve DNA repair and apoptosis, programmed cell death. The PARP enzymes have the capacity to make a polymer of ADP-ribose (PAR) from nicotinamide adenine dinucleotide (NADH in its reduced form).

Poly(ADP-ribose) polymerase-1 (PARP-1) is an example of a PARP enzyme which is able to bind damaged DNA and initiate the repair process upon recognition of DNA breaks caused by various genotoxic insults. Once bound to DNA, PARP-1 is activated and uses G-NAD⁺ to poly(ADP-ribosyl)ate proteins such as histones, transcription factors, and itself (in an automodification that leads to inactivation), thus markedly altering the overall size and charge of the modified protein (see FIG. 1). Sites for poly(ADP-ribose) (PAR) binding have been identified in numerous DNA-damage checkpoint proteins including tumor suppressor p53, DNA-ligase III, X-ray repair cross-complementing 1 (XRCC1), DNA-dependent protein kinase (DNA-PK), NF-κB, and telomerase, consistent with the role of PAR in the DNA repair pathway. The rate of PAR synthesis is directly proportional to the number of single and double strand breaks found in DNA, and while the amount of PAR may increase more than 100-fold in minutes immediately following DNA-damage, synthesis of such polymers is transient and closely regulated by poly(ADP-ribose) glycohydrolase (PARG), which cleaves PAR to ADP-ribose monomers.

The cytoprotective role of PARP-1 in response to DNA damaging agents has been studied and is supported by experiments with PARP-1-deficient cell lines. Accordingly, inhibition of PARP-1 with small molecules has proven to potentiate anticancer drugs, and initial studies have demonstrated that some BRCA-1-deficient tumor cells are extremely sensitive to PARP-1 inhibition. On the other hand, extreme DNA damage leads to PARP-1 overactivation and a severe depletion in cellular β-NAD⁺/ATP stores. The resulting loss of cellular energy can cause necrotic cell death. Thus, overactivation of PARP-1 has a cytotoxic effect, and PARP-1 inhibitors can prevent cell death caused by ischemic and reactive oxygen species-associated injury.

Including PARP-1 there are several members in the PARP family of enzymes with significant biomedical relevance, and the ability to detect and measure the activity of such enzymes is of great interest. There is keen desire for capabilities to screen for PARP modifiers, in particular for such modifiers which are inhibitory small molecules. Even though members of the PARP family have fascinating and fundamental cellular functions, little progress has been made in developing isozyme-specific PARP inhibitors. This search for potent and selective compounds is hampered by an inadequacy of suitable reagents and methods facilitating the detection and measurement of PARP enzymatic activity; moreover, there is a lack of high-throughput assays. Most commonly, until now PARP activity has been detected with radiolabeled NAD⁺, but other assays have been developed which employ antibodies, biotinylated NAD⁺, or fluorescence based quantitation of NAD⁺. A desirable PARP assay should be inexpensive, sensitive, rapid, and logistically simple, but methods involving specialized/radioactive reagents can be cost prohibitive and time consuming especially when testing a large number of compounds.

The present invention therefore originated out of the need to address the problem of how to detect and measure PARP enzyme activity. As embodiments of the present invention, we herein disclose compositions and methods including a novel colorimetric PARP substrate synthesized from β-NAD⁺ and a continuous assay to detect and kinetically monitor activity for PARP enzymes such as PARP-1, tankyrase-1, and VPARP.

### SUMMARY OF THE INVENTION

The invention broadly relates to the field of poly(ADP-ribose) polymerase (PARP) enzymes and colorimetric substrates therefore, including compositions, methods of detecting, methods of monitoring, and methods of screening.

In general the terms and phrases used herein have their art-recognized meaning, which can be found by reference to standard texts, journal references and contexts known to those skilled in the art. The following definitions are provided to clarify their specific use in the context of the invention.

The following abbreviations are applicable. PARP, Poly(ADP-ribose) polymerase; PAR, poly(ADP-ribose); PARG, poly(ADP-ribose) glycohydrolase; VPARP, vault poly(ADP-ribose) polymerase; tank-ad, GST fused active domain of tankyrase.

In an embodiment, the invention provides a compound having the formula CX-1:

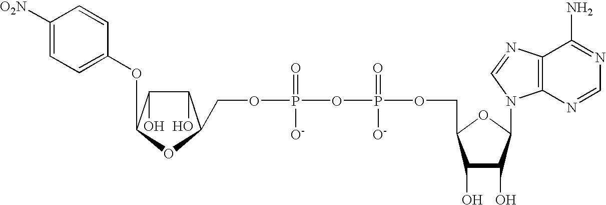

In an embodiment, the invention provides a method for detecting a poly(ADP-ribose) polymerase (PARP) enzyme activity, comprising providing a test sample putatively containing PARP enzymatic activity, reacting said sample with a colorimetric substrate, and observing a reaction product, thereby detecting said PARP enzyme activity. In an embodiment, said colorimetric substrate is a derivative of nicotinamide adenine dinucleotide (NAD). In an embodiment, said calorimetric substrate is formed from beta-NAD⁺ and para-nitrophenol. In an embodiment, said calorimetric substrate is compound CX-1.

In an embodiment, the PARP enzyme activity is an activity of a PARP enzyme selected from the group consisting of PARP-1, tankyrase-1 (PARP-5), and VPARP (PARP-4).

In an embodiment, the method comprises or further comprises kinetically monitoring said PARP enzyme activity, wherein said monitoring is achieved by performing a first observing step and at least a second observing of said reaction product, wherein said first and second observing steps are performed at different times. In an embodiment, the method comprises or further comprises kinetically monitoring said PARP enzyme activity, wherein said monitoring is achieved by providing at least a first test sample and a second test sample, independently reacting in separate reactions said samples with said calorimetric substrate, and independently observing said reaction products, wherein said observing occurs after different time periods of said reacting.

In an embodiment, the detecting further comprises providing a test substance in said test sample, wherein said test substance is a putative modifier of a PARP activity.

In an embodiment, the invention provides a method of screening for a substance putatively capable of modifying a PARP enzyme activity, comprising:

(a) providing a test material with putative PARP enzyme modification capability;
(b) providing a PARP enzyme;
(c) reacting in a test reaction said test material and said PARP enzyme with a PARP calorimetric substrate; and
(d) observing a reaction product of said reacting step; thereby screening for said material capable of modifying a PARP enzyme.

In an embodiment, the substance is putatively capable of inhibiting a PARP enzyme activity. In an embodiment, the substance is putatively capable of potentiating a PARP enzyme activity.

In an embodiment, the screening is high throughput screening and further comprises:

(e) providing at least a second test material; and
(f) in the first test reaction, independently in a second test reaction, or both in the first and second reactions; reacting said PARP enzyme with said PARP calorimetric substrate in the presence of said second test material; and
(g) if said second reaction is performed, observing a second reaction product.

In an embodiment, the invention provides a substrate compound capable of reacting specifically with a PARP enzyme, wherein said substrate is capable of forming a calorimetric product upon reaction with said PARP enzyme. In an embodiment, said PARP enzyme is PARP-1, tankyrase-1 (PARP-5), or VPARP (PARP-4).

In an embodiment, the invention provides a method of synthesizing a substrate for a PARP enzyme, comprising providing a nicotinamide adenine dinucleotide component, providing a nitrophenol component, and reacting said components, thereby generating said PARP enzyme substrate. In an embodiment, said substrate is a non-fluorescent substrate. In an embodiment, said substrate is a calorimetric substrate.

In an embodiment, the invention provides a composition comprising compound CX-1.

In an embodiment, the invention provides a kit for detecting a presence, absence, or level of a PARP enzyme activity, comprising a PARP-specific calorimetric substrate and at least one control sample, wherein said one control sample is either a positive control sample capable of exhibiting PARP enzyme activity or a negative control sample which lacks PARP activity. In an embodiment, the kit further comprises a modifier compound, wherein said modifier compound is capable of inhibiting a PARP enzyme activity or potentiating a PARP enzyme activity. In an embodiment, said colorimetric substrate is formed from a nicotinamide adenine dinucleotide component and a nitrophenol component. In an embodiment, said calorimetric substrate is compound CX-1.

In an embodiment of the kit, said PARP enzyme activity is an activity of a PARP enzyme selected from the group consisting of PARP-1, tankyrase-1 (PARP-5), and VPARP (PARP-4).

In an embodiment, the kit further comprises a solvent for said clorimetric substrate.

In an embodiment, the kit further comprises a second control sample, wherein said second control sample is a negative control sample if the first control sample is a positive control sample, and vice versa.

In an embodiment, a compound and/or composition is isolated or purified.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE FIGURES

FIG. 1. Synthesis of poly(ADP-ribose) onto glutamic acid residues of protein acceptors catalyzed by PARP enzymes, producing nicotinamide as a byproduct.

FIG. 2. FIG. 2(A) illustrates an overview of the elegant reaction scheme which uses para-nitrophenol to convert NAD⁺ to a useful colorimetric substrate for PARP enzymes. FIG. 2(B) illustrates kinetic data for tankyrase-1 obtained using the ADP-ribose-pNP PARP substrate. Analogous curves for PARP-1 and VPARP were also generated.

FIG. 3. FIG. 3 illustrates IC₅₀ curves for PARP-1, tankyrase-1 and VPARP.

FIG. 4. FIG. 4A, Calibration Curve for p-nitrophenol. FIG. 4B, Observance of ADP-ribose-pNP in PARP assay buffer (50 mM Tris, 10 mM MgCl2, pH 8.0) at 405 nm. ADP-ribose-pNP is stable for more than 24 hours at room temperature in PARP assay buffer as observed by both NMR and absorbance (at 405 nm) measurements.

FIG. 5. FIG. 5A, Results of observing PARP-1 kinetics with ADP-ribose-pNP. FIG. 5B, VPARP kinetics with ADP-ribose-pNP.

FIG. 6 illustrates the ¹H spectrum of ADP-ribose-pNP in d₆-DMSO/D₂O.

FIG. 7 illustrates the ¹³C spectrum of ADP-ribose-pNP in d₆-DMSO/D₂O.

FIG. 8 illustrates the optical rotation data of ADP-ribose-pNP ([α]=14.66).

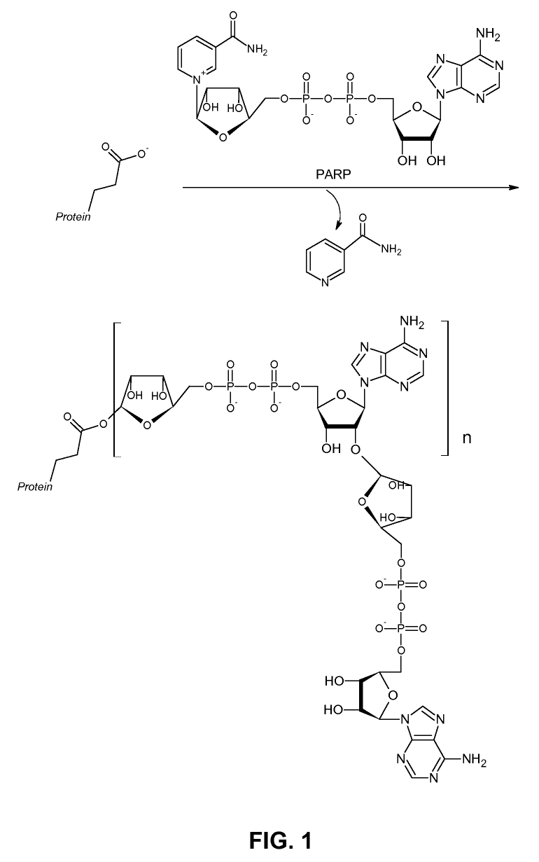

### DETAILED DESCRIPTION OF THE INVENTION

The invention may be further understood by the following non-limiting examples.

### EXAMPLE 1

### Synthesis of Novel Colorimetric Substrate for PARP Enzymes

PARPs comprise a large family of about 18 putative isozymes. While basic enzymatic function and biochemistry has been characterized for at least six members of this family, much work remains to be done in this area. Although PARP-1 accounts for more than 90% of PAR synthesis upon DNA damage, it is now known that biopolymer synthesis by various other PARPs is critical in a variety of cellular processes. Particularly intriguing are the functions of tankyrase-1 (PARP-5) and VPARP (PARP-4). Unlike PARP-1, tankyrase-1 is not activated by DNA damage. About 10% of this protein is recruited to telomeres, and it has been shown that overexpression of tankyrase-1 can lengthen telomeres through its poly(ADP-ribosyl)ation of TRF-1. VPARP is most commonly found associated with vault particles, but can also localize to the nucleolus, nuclear spindle, or nuclear pores.

The ability to study PARP enzymes would be greatly augmented by enhanced capabilities for the detection and measurement of enzymatic activity. Furthermore, the ability to screen and identify PARP inhibitors including isozyme-specific PARP inhibitors is facilitated by the present invention. We now report the development of a continuous assay which utilizes a novel colorimetric PARP substrate to kinetically monitor PARP enzymatic activity. In preferred embodiments, the novel substrate is useful in connection with detecting PARP-1, tankyrase-1, and VPARP activity. As herein disclosed, the substrate is easily synthesized from M-NAD⁺.

All PARP isozymes utilize β-NAD⁺ to synthesize ADP-ribose polymers, producing nicotinamide as a byproduct. By exchanging the nicotinamide moiety of NAD⁺ for a colorimetric leaving group, a substrate suitable for a continuous kinetic PARP assay is provided. The identification of a way to develop such a suitable substrate was in part due to our recognition that PARP-1 can utilize biotinylated NAD⁺ to synthesize poly(ADP-ribose). The use of commercially available β-NAD⁺ as a starting material can greatly simplify the synthesis and scaled-up production of this substrate. Very little literature precedent exists for the chemical modification of NAD⁺; basic methanolysis is possible and yields a 3.7:1 mixture of β:α anomers, whereas methods employing the NADase/NAD⁺ enzymatic system provide N- and O-(ADP-ribosyl)ation products, albeit in low yield and limited substrate scope. We adapted a strategy for the preparation of ADP-ribose-pNP upon recognizing that syntheses of cyclic-ADP-ribose analogs have utilized nucleophilic metal halides in the presence of triethylamine to successfully and stereoselectively cyclize NAD⁺.

The compound ADP-ribose-pNP (designated CX-1) was synthesized directly from commercially available β-NAD⁺ by stirring with sodium bromide and triethylamine in DMSO at 70° C. for 2 hours (see Scheme 1).

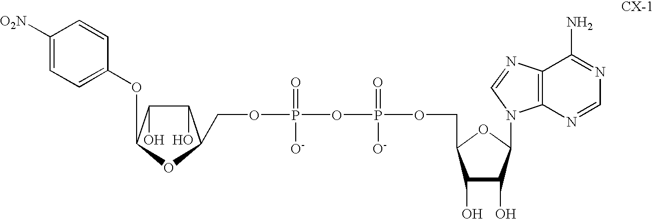

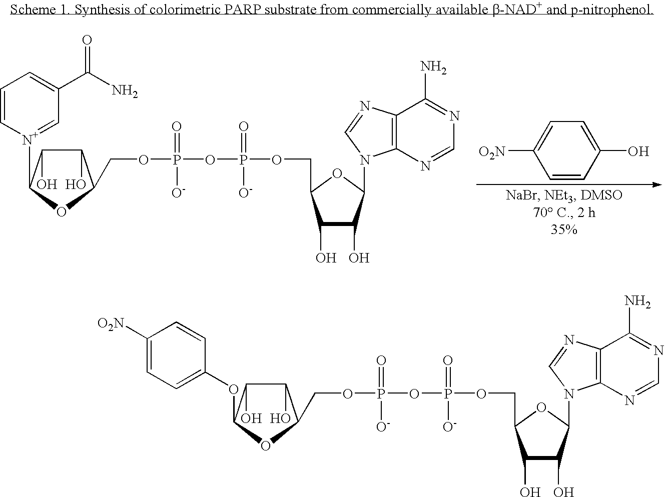

This reaction can easily be performed on the 250-500 mg scale, with isolated yields of 35%. Purification involves removal of excess DMSO under reduced pressure and reverse phase column chromatography. The absolute configuration at the anomeric position was assigned on the basis of coupling constants and nOe experiments, both of which indicate the product is the β-anomer of ADP-ribose-pNP (see Supporting Information). In addition, similar reactions can produce products of the beta-configuration (see Yamada et al., 1994). Control experiments revealed that ADP-ribose-pNP is stable in aqueous buffer under PARP assay conditions (50 mM Tris, 10 mM MgCl₂, pH 8.0, room temperature) for at least 24 hours, and is generally stable between pH 4 and 8.

### EXAMPLE 2

### Development of Continuous Assay for Monitoring PARP Enzymatic Activity

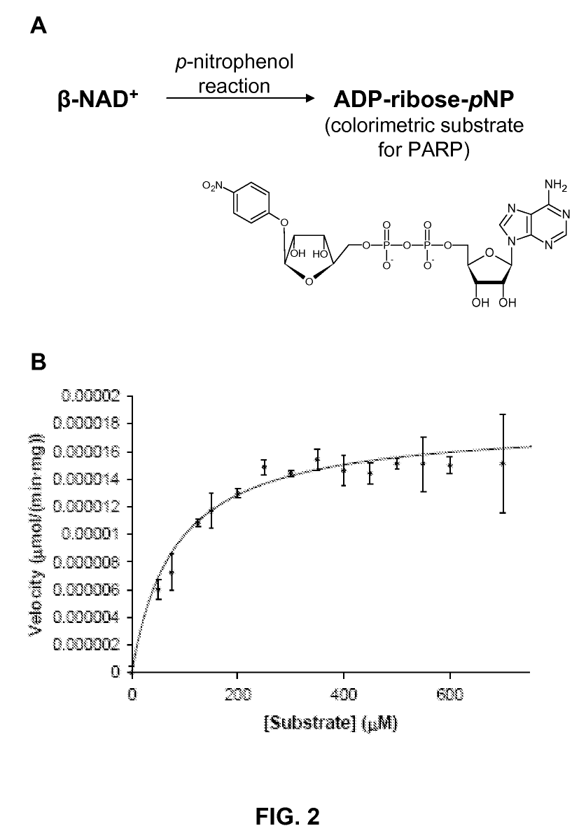

With the calorimetric substrate ADP-ribose-pNP in hand, a continuous calorimetric assay for PARP activity was developed and the kinetic parameters for three PARP isozymes were determined. In a 96-well plate, a range of concentrations of ADP-ribose-pNP in PARP assay buffer were incubated with either PARP-1 (DNase digested DNA was added to activate PARP-1), tankyrase-1 (refers to “active domain,” see Supporting Information), or VPARP (also refers to the “active domain”), and the optical density at 405 nm was measured every 60 seconds over a 2 hour time period. Change in absorbance was assessed in triplicate, and blanks containing 0 to 700 μM ADP-ribose-pNP in PARP assay buffer were also measured over the same time period. The absorbance of a range of p-nitrophenol concentrations was determined at 405 nm, and the slope of this calibration curve (see Supporting Information) was used to convert the absorbencies to moles of product generated; in this way the kinetic parameters for PARP-1, tankyrase-1, and VPARP were calculated (see Table 1 and FIG. 2).

TABLE 1
Comparison of kinetic data for PARP-1, tankyrase-1 and VPARP as
reported in the literature and with the substrate, ADP-ribose-pNP.

| | | ARP-1 | | tankyrase-1 | |
|---|---|---|---|---|---|
| Parameter | PARP-1 | literature^{[a–c]} | tankyrase-1 | literature[d] | VPARP |
| kcat (s-1) | 0.025 | 0.41 | 1.88 × 10⁻⁵ | 0.71 | 2.18 × 10⁻⁶ |
| KM (μM) | 151 | 59–278 | 82 | 1500 | 46 |
| Vmax | 1.30 × 10⁻³ | 0.2–2.4 | 1.81 × 10⁻⁵ | — | 2.03 × 10⁻⁶ |
| μmol/(min · mg) | | | | | |

^{[a]}Kawaici M et a., J. Biol. Chem. 1981, 256, 9483;
^{[b]}Beneke S et al., Exp. Gerontol. 2000, 35, 989;
^{[c]}Banasik M et al., Acta Neurobiol. Exp. 2004, 64, 4;
[d]Rippmann JF et a., J. Mol. Biol. 2003, 325, 1107.

Consistent with data in the literature, PARP-1 has the largest K_{M} and Vₘₐₓ (151 μM and 1.30×10⁻³ μmol/(min·mg) respectively) followed by tankyrase-1 (82 μM and 1.81×10⁻⁵ μmol/(min·mg)) and VPARP (46 μM and 2.03×10⁻⁶ μmol/(min·mg)). While exact kinetic parameters for PARP-1 can vary with the assay used, for PARP-1 the K_{M} value with ADP-ribose-pNP is consistent with that of the natural β-NAD⁺ substrate, while the Vₘₐₓ is approximately 100 fold lower with the calorimetric substrate (see Table 1). For tankyrase-1, the K_{M} is approximately 18-fold lower and the Vₘₐₓ is significantly lower as compared to the values reported in the literature with β-NAD⁺ as a substrate. To our knowledge, no data is available on the kinetic parameters of VPARP with β-NAD⁺. Control experiments in which bovine serum albumin was incubated with the ADP-ribose-pNP substrate produced no signal at 405 nm, indicating that this substrate is not generally processed by proteins in a non-specific manner (see Supporting Information).

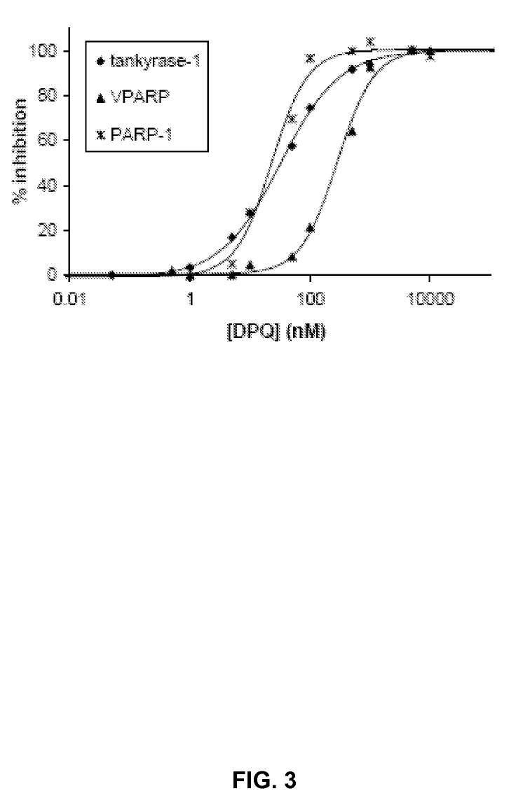

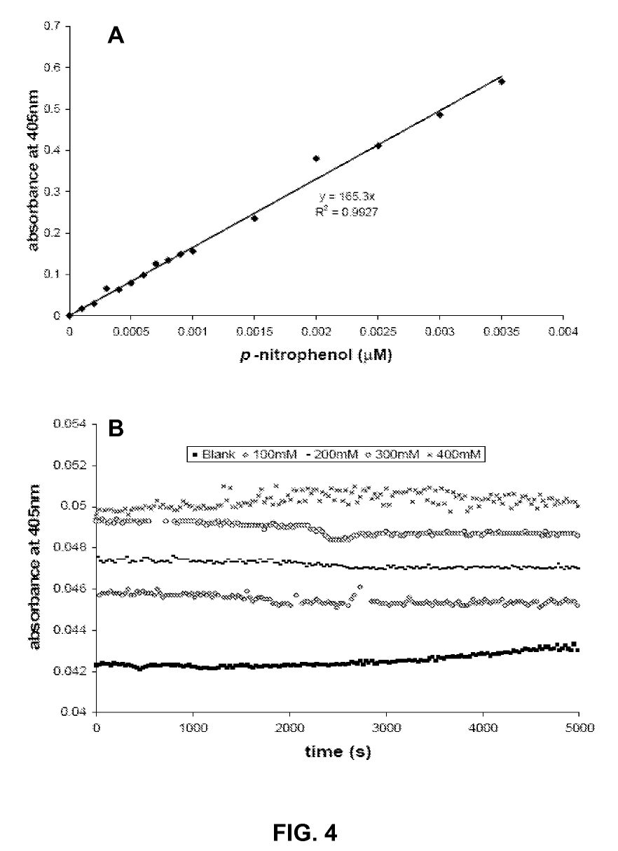

Next, in order to demonstrate the potential of this assay to identify isozyme-specific PARP inhibitors, we utilized ADP-ribose-pNP to determine the IC₅₀ value of the known PARP-1 inhibitor 3,4-Dihydro-5-[4-(1-piperidinyl)butoxyl]-1 (2H)-isoquinolinone (DPQ)[37] with PARP-1, tankyrase-1, and VPARP. For these measurements, concentrations of DPQ ranging from 0.05 nM to 10 μM were added to a 96-well plate containing PARP assay buffer and ADP-ribose-pNP and the absorbance at 405 nm was measured in triplicate. As shown in FIG. 3, DPQ has similar IC50 values for all of the PARP isozymes tested, though it has a slightly lower IC50 value for PARP-1 (23 nM) and tankyrase-1 (33 nM) as compared to VPARP (281 nM). Literature reports of the IC50 value for DPQ with PARP-1 range from 40 nM to 3500 nM[37-39] It should be noted that IC50 values have never been determined for any compounds with VPARP and tankyrase-1.

Utilizing ADP-ribose-pNP as a calorimetric substrate, we have developed a simple, sensitive, and inexpensive kinetic assay for assessing activity of the PARP family of enzymes. This novel substrate has been used to determine the kinetic parameters for PARP-1, tankyrase-1, and VPARP, and it has been employed to obtain IC50 values for a small molecule inhibitor. With this new tool for elucidating PARP activity, we now have the ability to gain further understanding of the kinetic activity of the diverse PARP family. As ADP-ribose-pNP lends itself easily to milligram-scale synthesis, testing of large libraries to find isozyme-specific inhibitors of these enzymes should be a straightforward task which will provide even more information about the specific biochemical function of each isozyme and potentially lead to targeted therapies.

### EXAMPLE 3

### Supporting Information

Reagents. High specific activity PARP-1 was purchased from Trevigen. β-NAD⁺, p-nitrophenol, and 3,4-dihydro-5-[4-(1-piperidinyl)butoxy]-1 (2H)-iso-quinolinone (DPQ) were purchased from Sigma-Aldrich. DMSO and triethylamine were distilled and stored over molecular sieves prior to use. PARP assay buffer consisted of 50 mM Tris, 2 mM MgCl2 at pH 8.0, and was freshly prepared before each experiment.

General Methods. ¹H NMR and ¹³C NMR spectra were recorded on a Varian Unity 500 MHz, ¹H (125.7 MHz, ¹³C) spectrometer or a Varian Unity Inova 500NB. Chemical shifts are reported in parts per million (ppm), and multiplicities are denoted as s (singlet), d (doublet), t (triplet), m (multiplet), and br (broad). Spectra were referenced to d6-DMSO (1H 2.49 ppm, ¹3C 39.5 ppm) or D2O (1H 4.65 ppm). Mass spectra were obtained by the University of Illinois Mass Spectrometry Center and the data is reported as m/z. Analytical thin layer chromatography (TLC) was performed on precoated silica gel plates with indicator. Spots were visualized by UV light.

Synthesis of ADP-ribose-pNP. p-Nitrophenol (525 mg, 3.77 mmol, 5 eq.), NaBr (3.7 g, 30.6 mmol, 40.6 eq) and β-NAD⁺ (500 mg, 0.753 mmol, 1 eq.), were dissolved in 5 mL of freshly distilled DMSO. To this solution was added triethylamine (250 μL), and the mixture was stirred at 70° C. for 2 h under an atmosphere of nitrogen. After the solution was cooled, excess DMSO was removed under reduced pressure, and the remaining residue was dissolved in a small amount of water and purified on an Alltech high capacity C18 Extract-clean column (5000 mg/25 mL) in 3 portions, using water as the elutant. Impure fractions were kept and resubmitted to reverse phase column chromatography until all fractions were pure, which involved approximately 4-5 additional columns. The desired ADP-ribose-pNP was obtained in 35% yield. It should be noted that Amberlite XAD-7 nonionic polymeric adsorbent (Sigma-Aldrich) can also be utilized for purification of ADP-ribose-pNP, although additional reverse phase columns are needed to completely remove the salt that is present.

R_{f} (7:3 isopropanol: 0.2% NH₄OH (aq.)) 0.73

¹H NMR (500 MHz, D₂O/d₆-DMSO)

δH [ppm]=3.92 (2H, dd, J=4.25 Hz, J=9 Hz, H-14), 4.07 (2H, br, H-13), 4.11 (1H, dd, J=2.5 Hz, J=6.0 Hz, H-12), 4.18 (1H, br, H-11), 4.20 (1H, br, H-10), 4.24 (1H, dd, J=4.5 Hz, J=4.5 Hz, J=6.0, H-9) 4.30 (1H, dd, J=4.25 Hz, J=4.25 Hz, H-8), 4.45 (1H, dd, J=5.25 Hz, J=5.25 Hz, H-7), 5.47 (1H, d, J=4.5 Hz, H-6), 5.79 (1H, d, J=5 Hz, H-5), 6.69 (2H, d, J=2.25 Hz, H-4), 7.65 (2H, d, J=2 Hz, H-3), 7.75 (1H, s, H-2), 8.14 (1H, s, H-1)

¹³C NMR (500 MHz, D₂O/d₆-DMSO)

δC [ppm]=66.95 (C-13), 66.46 (C-14), 70.43 (C-12), 71.04 (C-8), 71.98 (C-9), 75.20 (C-7), 84.33 (C-10), 85.40 (C-11), 87.60 (C-5), 101.00 (C-6), 116.90 (C-4), 118.81 (C-15), 126.19 (C-3), 140.14 (C-1), 142.07 (C-15), 149.16 (C-6), 153.17 (C-2), 155.72 (C-17), 162.41 (C-18)

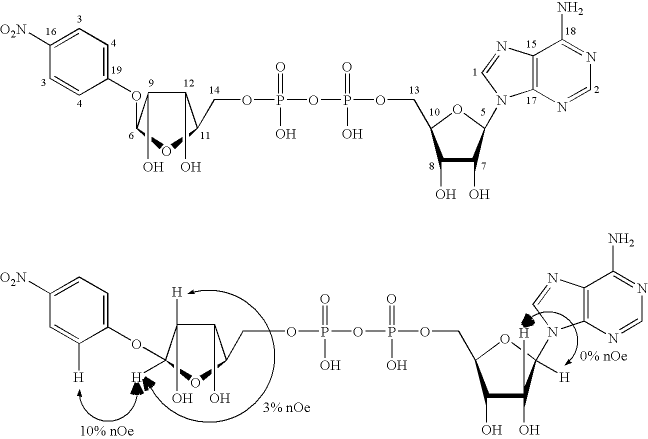

MS m/z (M⁺) calculated for C₂₁H₂₆O₁₆P₂Na 703.0778, HRMS found 703.0784

Calibration curve with p-nitrophenol. 100 μL of 0 to 35 μM p-nitrophenol in PARP assay buffer was added in triplicate to the wells of a Falcon UV-Vis transparent 96-well plate, and the absorbance at 405 nm was read on a SpectraMax Plus (Molecular Devices). The results were averaged and corrected to 0.

Expression of VPARP. The plasmid with the catalytic domain of VPARP, pET28b-p193cat, was the kind gift of Dr. Valerie Kickhoefer. An overnight culture of pET28b-p193cat in E. coli Rosetta (EMD Biosciences) grown in Luria Broth (LB)/kanamycin (100 μg/mL)/chloramphenicol (37.5 μg/mL) was used to inoculate an 8 L L/kanamycin (100 μg/mL)/chloramphenicol culture. This 8 L culture was incubated at 37° C., 225 rpm until the OD₆₀₀ reached 0.8. At this point isopropyl-β-D-thiogalactopyranoside (IPTG) was added to the culture to a final concentration of 300 μM, and incubation at 37° C., 225 rpm was continued for a period of 1 hour. The cells were then harvested by centrifugation at 5000×g for 8 minutes. The supernatant was discarded and the pellet was resuspended in 30 mL Binding Buffer (50 mM Tris pH 8.0, 100 mM NaCl, 5 mM imidazole). Cells were lysed by sonication. The lysate was centrifuged at 35,000×g for 30 minutes. The supernatant was separated from the pellet and incubated with 3 mL of Ni-NTA resin slurry (Qiagen) for 1 hour at 4° C. After this batch loading process, the supernatant and Ni-NTA agarose-resin was loaded onto a 15 mL column. The column was washed with 10 mL cold Binding Buffer, 10 mL Wash Buffer (50 mM Tris pH 8.0, 300 mM NaCl, 20 mM imidazole), and VPARP was eluted with 10 mL cold Elution Buffer (identical to Wash Buffer except 250 mM imidazole was added). All elution fractions were analyzed for the presence of protein using the Bradford dye reagent (Bio-rad). All samples containing protein were combined and concentrated using the Centricon centrifugal concentration device, 30,000 molecular weight cutoff (MCWO) (Millipore). The protein molecular weight was confirmed by SDS-PAGE analysis.

Expression and purification of tankyrase-1 active domain. The full-length tankyrase gene was the kind gift of Dr. Susan Smith. The SAM and PARP domain of tankyrase was subcloned into pGEX-5X-1 (Amersham Biosciences). This GST fused active domain of tankyrase (tank-ad) was transformed into E. coli Rosetta cells (EMD Biosciences) followed by growth overnight in 250 mL of Luria Broth (LB). Plasmid and bacterial selections were done using 50 μg/mL of ampicillin and 37.5 μg/mL of chloramphenicol respectively. The overnight culture was seeded into 12 L of LB under the same selection conditions as the overnight culture. Bacteria were grown at 37° C. with shaking at 250 rpm until the OD₆₀₀ was 0.7. At this point isopropyl-β-D-thiogalactopyranoside (IPTG) was added to the culture to a final concentration of 500 μM. Bacteria were harvested by centrifugation at 5000 rpm for 7 minutes followed by re-suspension in 75 mL of phosphate buffered saline (PBS) containing 1% Triton X-100, 2 mM EDTA, and 2 mM PMSF. Cells were lysed by sonication and then centrifuged at 17,500 rpm for 30 minutes. The supernatant was separated from the pellet and 2.66 mL of glutathione sepharose 4B (Amersham Biosciences) that had been washed with 20 mL of PBS was added to the supernatant and incubated for 3 hours at 4° C. with agitation. The supernatant and resin were then passed through a 1.5 cm fritted glass column. The resin was washed with 50 mL of PBS, and protein was eluted by mixing 4 mL of 20 mM reduced glutathione in 50 mM Tris-HCl, pH 8.0 with the resin and letting it incubate for 1 hour at 4° C. After the eluted protein had passed through the column it was placed in a 15 mL spin concentrator with a 5000 MWCO (Centricon). By successive 20 minute centrifugations at 5000×g, the glutathione buffer was exchanged for PARP assay buffer (50 mM Tris-HCl, 10 mM MgCl2, pH 8.0), and the protein was concentrated until the volume was 2.2 mL. The protein concentration was estimated spectroscopically (using an extinction coefficient at 280 nm of 65835 M⁻¹ cm⁻¹ generated by the program ProtParam (http://au.expasy.org)), and found to be approximately 75 μM.

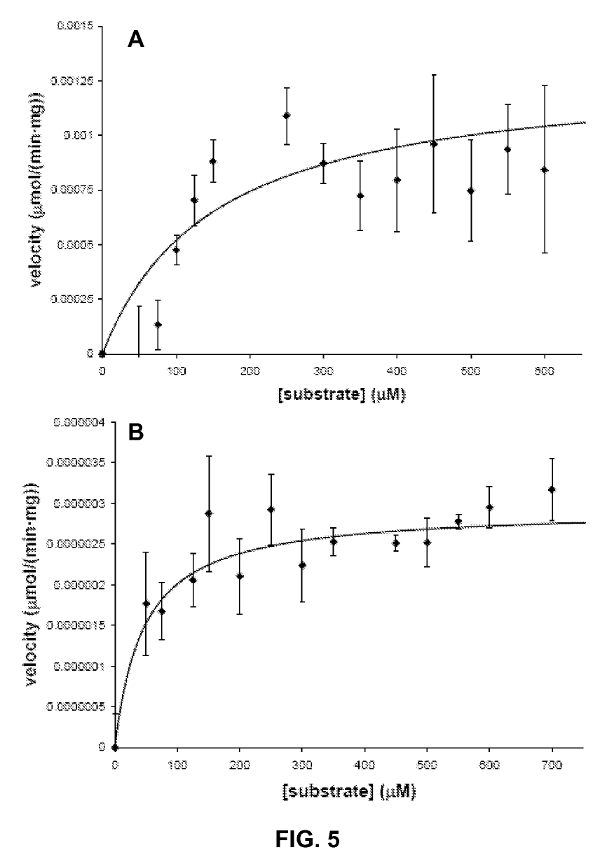

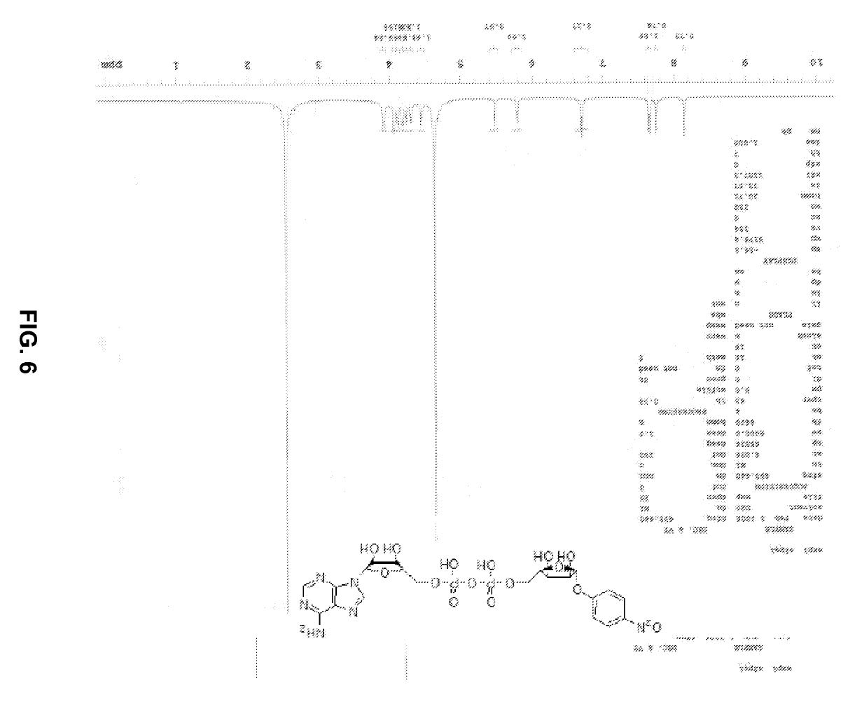

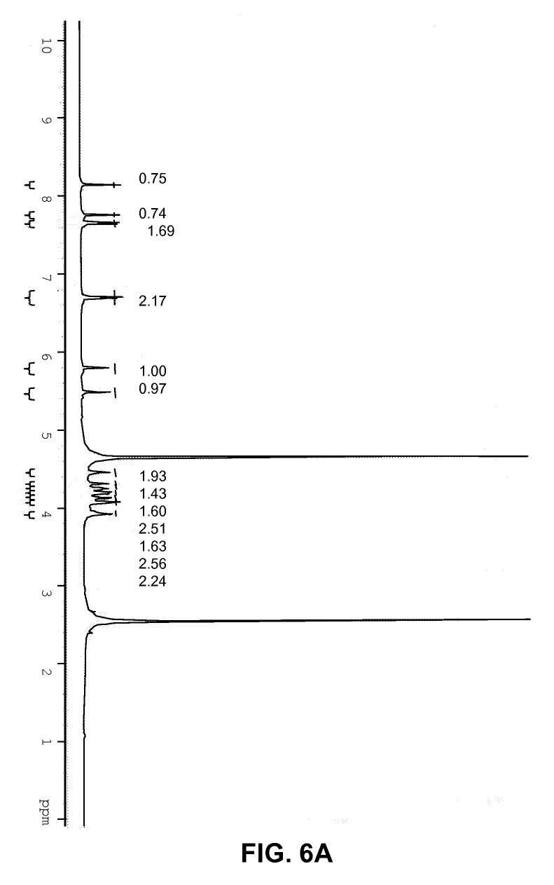

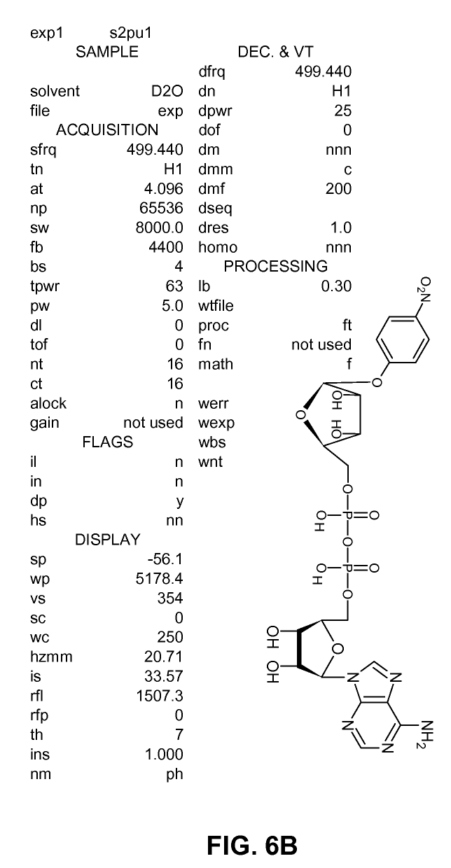

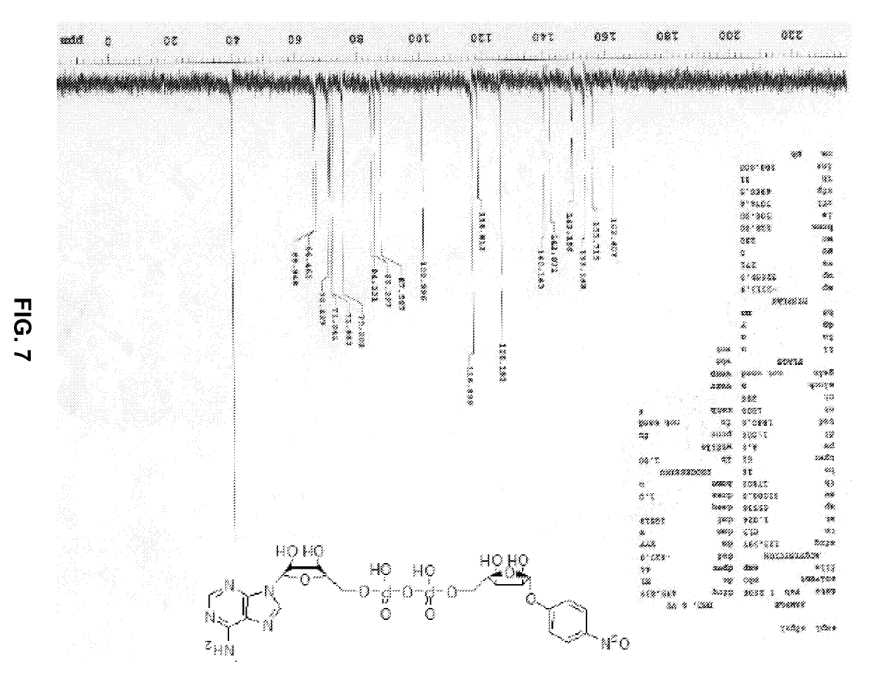

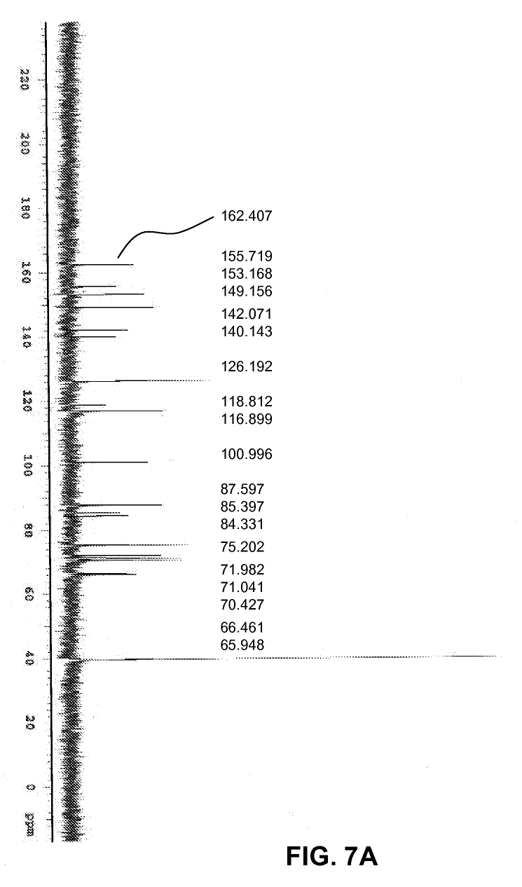

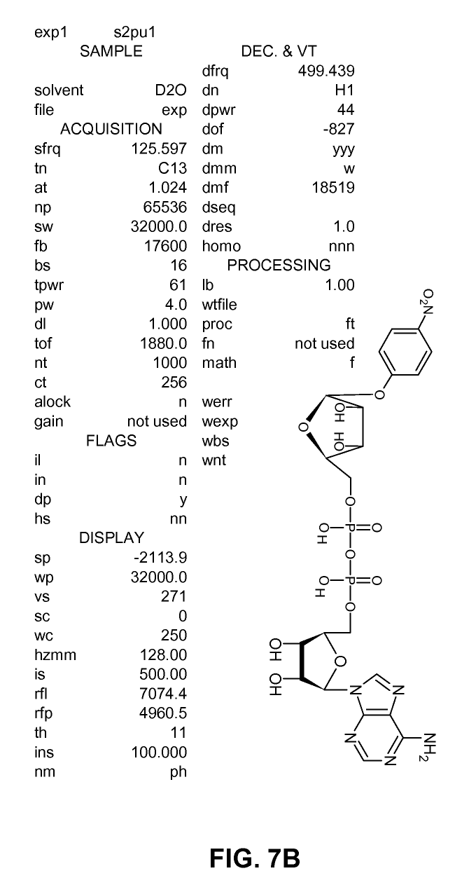

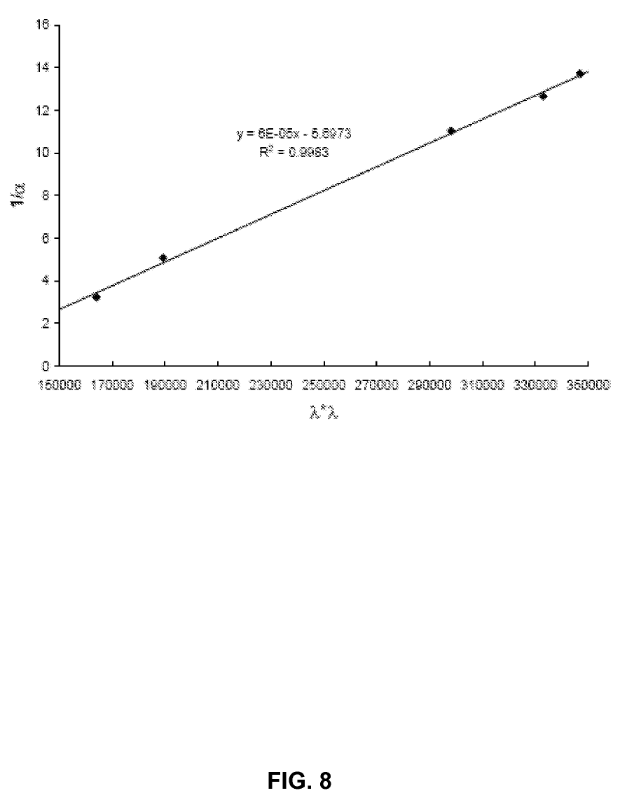

Kinetics of VPARP and Tankyrase-1. The ADP-ribose-pNP colorimetric substrate was diluted from a 10 mM stock in PARP assay buffer to 1142.9 μM. From this stock, dilutions into PARP assay buffer were made in 15×1.7 mL tubes so that the volume was 490 μL. This was enough to perform the experiment in triplicate, with additional wells (no protein) to serve as blanks for each substrate concentration. The concentration of substrate in each tube was made such that 70 μL from each tube could be added to the wells of a 96 well plate. When each well was brought to the final volume of 100 μL, the desired final concentration was reached. See FIG. 2B and FIG. 5B for tank-ad and VPARP substrate concentrations, respectively. Concentrations ranged from 0 to 700 μM with a finer distribution at lower substrate concentrations.

The plate was divided in half with protein added to half the wells and the other half serving as blanks containing only PARP assay buffer and substrate. After adding 70 μL of substrate from each tube to 3 wells on the protein side or 3 wells of the blank side, 30 μL of PARP assay buffer was added to all the wells of the blank side. Next, 30 μL of the enzyme was added to each of the wells on the protein half of the plate to initiate the reaction, with a final concentration of tankyrase per well of 21.8 μM and the final concentration of VPARP per well of 19.8 μM. The plate was then read on a SpectraMax plus 384 UV/Vis plate reader (Molecular Devices), at 405 nm for 2 hours with observations performed once every minute. Mixing was conducted between observations.

Preparation of damaged DNA for PARP-1. 500 μL of Herringsperm DNA (10 mg/ml) was added to 100 mL DNase buffer (400 mM Tris-HCl, 100 mM MgSO₄, and 10 mM CaCl₂, pH 8.0), along with 50 μL of DNase (1 unit/mL). This solution was first incubated at 37° C. for 1 minute and then heated at 90° C. for 20 minutes to inactivate the DNase. Damaged DNA was used without any further purification.

Kinetics of PARP-1. A 160 μL volume of PARP-1 (0.45 mg/mL) was premixed with 262.4 μL of the damaged herringsperm DNA and 2777.6 μl of PARP assay buffer. This mixture (known as activated PARP in assay buffer) was allowed to equilibrate on ice for 4 hours. The ADP-ribose-pNP colorimetric substrate was diluted from a 5000 μM stock in PARP assay buffer directly into the wells of a 96 well plate. For the first half of the plate a 50 μL volume of each substrate concentration was initially prepared in triplicate (double the concentration used in the assay) by diluting 0 to 14 μL of substrate in PARP assay buffer (5000 μM stock) into 50 to 36 μL of PARP assay buffer. For the second half of the plate, a 100 μL volume of each substrate concentration was prepared from a 5000 μM stock of ADP-ribose-pNP. This half of the plate served as substrate blanks. Finally, to the first half of the plate (which already contained 50 μL of substrate concentrations ranging from 0 to 700 μM) 50 μL of the activated PARP in assay buffer was added to each well. The final concentration of PARP-1 per well was 9.9 nM. The plate was then read on a SpectraMax plus UV/Vis plate reader (Molecular Devices), at 405 nm for 2 hours, reading once every minute with mixing between reads. Results of PARP-1 kinetics are shown in FIG. 5A.

Control experiment with BSA. To determine that substrate cleavage is specific to the PARP family of enzymes, 16 μL of BSA was added to 81.5 μL of PARP assay buffer in a 96 well plate so that the final concentration was 20 μM in protein. The experiment was performed in triplicate along with controls that contained PARP assay buffer only. To both the wells containing only buffer or buffer and BSA, 2.5 μL of a 10 mM stock of the substrate was added to start the reaction. The plate was monitored at 405 nm for 2 hours.

Determination of IC₅₀ values for PARP inhibitors. To determine IC₅₀ values of the PARP inhibitors, 22.5 μL of PARP assay buffer containing varying concentrations of DPQ and 20 μg/mL DNase activated DNA (for PARP-1 only) were added into the wells of a 384-well plate. A 22.5 μL volume of PARP at a concentration of 5 μg/mL PARP-1, 40 μg/mL VPARP and 40 μg/mL tankyrase-1 in PARP assay buffer was added. The reaction was initiated by the addition of 5 μL of a 2.5 mM solution of ADP-ribose-pNP in PARP assay buffer, bringing the final concentration to 2.5 μg/mL PARP-1 with 10 μg/mL DNA or 20 μg/mL VPARP or 20 μg/mL tankyrase-1, 250 μM ADP-ribose-pNP with varying concentrations of inhibitors in a total volume of 50 μL. The plate was then read every 2 minutes for 2 hours at 405 nm on a SpectraMax 384 plate reader (Molecular Devices). The average value of control wells containing only substrate was set as 0% PARP activity, while the average value of control wells containing PARP and substrate (but no inhibitor) was set as 100% PARP activity. The values obtained from the various concentrations of inhibitors were converted to a percentage of PARP activity and plotted.

Data analysis. Data from the plate reader was imported into Excel where appropriate subtractions were made and the data was plotted. Graphs were analyzed using Table Curve 2D. A p-nitrophenol standard curve was fitted with a least squares linear model, kinetic curves were fitted with a second order formation model (equation 8108) and inhibitor curves were fitted with a logistic dose response curve (equation 8013).

### STATEMENTS REGARDING INCORPORATION BY REFERENCE AND VARIATIONS

All references mentioned throughout this application, for example patent documents including issued or granted patents or equivalents; patent application publications; unpublished patent applications; and non-patent literature documents or other source material; are hereby incorporated by reference herein in their entireties, as though individually incorporated by reference. In the event of any inconsistency between cited references and the disclosure of the present application, the disclosure herein takes precedence. Some references provided herein are incorporated by reference to provide information, e.g., details concerning sources of starting materials, additional starting materials, additional reagents, additional methods of synthesis, additional methods of analysis, additional biological materials, additional cells, and additional uses of the invention.

All patents and publications mentioned herein are indicative of the levels of skill of those skilled in the art to which the invention pertains. References cited herein can indicate the state of the art as of their publication or filing date, and it is intended that this information can be employed herein, if needed, to exclude specific embodiments that are in the prior art. For example, when composition of matter are claimed herein, it should be understood that compounds known and available in the art prior to Applicant's invention, including compounds for which an enabling disclosure is provided in the references cited herein, are not intended to be included in the composition of matter claims herein.

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Thus as used herein, comprising is synonymous with including, containing, having, or characterized by, and is inclusive or open-ended. As used herein, “consisting of” excludes any element, step, or ingredient, etc. not specified in the claim description. As used herein, “consisting essentially of” does not exclude materials or steps that do not materially affect the basic and novel characteristics of the claim (e.g., relating to the active ingredient). In each instance herein any of the terms “comprising”, “consisting essentially of” and “consisting of” may be replaced with either of the other two terms, thereby disclosing separate embodiments and/or scopes which are not necessarily coextensive. The invention illustratively described herein suitably may be practiced in the absence of any element or elements or limitation or limitations not specifically disclosed herein.

Whenever a range is disclosed herein, e.g., a temperature range, time range, composition or concentration range, or other value range, etc., all intermediate ranges and subranges as well as all individual values included in the ranges given are intended to be included in the disclosure. This invention is not to be limited by the embodiments disclosed, including any shown in the drawings or exemplified in the specification, which are given by way of example or illustration and not of limitation. It will be understood that any subranges or individual values in a range or subrange that are included in the description herein can be excluded from the claims herein.

The invention has been described with reference to various specific and/or preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be employed in the practice of the invention as broadly disclosed herein without resort to undue experimentation; this can extend, for example, to starting materials, biological materials, reagents, synthetic methods, purification methods, analytical methods, assay methods, and biological methods other than those specifically exemplified. All art-known functional equivalents of the foregoing (e.g., compositions, methods, devices, device elements, materials, procedures and techniques, etc.) described herein are intended to be encompassed by this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, preferred embodiments, and optional features, modification and variation of the concepts herein disclosed may be resorted to by those skilled in the art, and that such modifications and variations are considered to be within the scope of this invention as defined by the appended claims.

### REFERENCES

A. Bürkle, ChemBioChem 2001, 2, 725.
S. Cuzzocrea, Z. Q. Wang, Pharmacol. Res. 2005, 52, 100.
G. de Murcia, J. Menissier de Murcia, Trends Biochem. Sci. 1994, 19, 172.
D. D'Amours, S. Desnoyers, I. D'Silva, G. G. Poirier, Biochem. J. 1999, 342 (Pt 2), 249.
W. M. Tong, U. Cortes, Z. Q. Wang, Biochim. Biophys. Acta 2001, 1552, 27.
M. E. Bonicalzi, J. F. Haince, A. Droit, G. G. Poirer, Cell. Mol. Life Sci. 2005, 62, 739.
M. L. Meyer-Ficca, R. G. Meyer, D. L. Coyle, E. L. Jacobson, M. K. Jacobson, Exp. Cell. Res. 2004, 297, 521.
L. Davidovic, M. Vodenicharov, E. B. Affar, G. G. Poirier, Exp. Cell Res. 2001, 268, 7.
F. Le Page, V. Schreiber, C. Dherin, G. De Murcia, S. Boiteux, J. Biol. Chem. 2003, 278, 18471.
C. A. Delaney, L. Z. Wang, S. Kyle, A. W. White, A. H. Calvert, N. J. Curtin, B. W. Durkacz, Z. Hostomsky, D. R. Newell, Clin. Cancer Res. 2000, 6, 2860.
P. Jagtap, C. Szabo, Nat. Rev. Drug Discov. 2005, 4, 421.
H. E. Bryant, N. Schultz, H. D. Thomas, K. M. Parker, D. Flower, E. Lopez, S. Kyle, M. Meuth, N. J. Curtin, T. Helleday, Nature 2005, 434, 913.
H. Farmer, N. McCabe, C. J. Lord, A. N. Tutt, D. A. Johnson, T. B. Richardson, M. Santarosa, K. J. Dillon, I. Hickson, C. Knights, N. M. Martin, S. P. Jackson, G. C. Smith, A. Ashworth, Nature 2005, 434, 917.
J. L. Sims, S. K. Berger, N. A. Berger, Biochemistry 1983, 22, 5188-5194.
I. U. Schraufstatter, P. A. Hyslop, D. B. Hinshaw, R. G. Spragg, L. A. Sklar, C. G. Cochrane, Proc. Natl. Acad. Sci. USA 1986, 83, 4908.
C. Szabo, V. L. Dawson, Trends Pharmacol. Sci. 1998, 19, 287.
C. Thiemermann, J. Bowes, F. P. Myint, J. R. Vane, Proc. Natl. Acad. Sci. USA 1997, 94, 679.
J. Zhang, S. Lautar, S. Huang, C. Ramsey, A. Cheung, J. H. Li, Biochem. Biophys. Res. Commun. 2000, 278, 590.
J. C. Ame, C. Spenlehauer, G. de Murcia, Bioessays 2004, 26, 882.
P. O. Hassa, S. S. Haenni, M. Elser, M. O. Hottiger, Microbiol. Mol. Biol. Rev. 2006, 70, 789.
S. Smith, Trends Biochem. Sci. 2001, 26, 174.
J. F. Haince, M. Rouleau, M. J. Hendzel, J. Y. Masson, G. G. Poirier, Trends Mol. Med. 2005, 11, 456.
S. Smith, I. Giriat, A. Schmitt, T. de Lange, Science 1998, 282, 1484.
S. Smith, T. de Lange, Curr. Biol. 2000, 10, 1299.
V. A. Kickhoefer, A. C. Siva, N. L. Kedersha, E. M. Inman, C. Ruland, M. Streuli, L. H. Rome, J. Cell Biol. 1999, 146, 917.
A. van Zon, M. H. Mossink, M. Schoester, A. B. Houtsmuller, G. L. Scheffer, R. J. Scheper, P. Sonneveld, E. A. Wiemer, J. Cell Sci. 2003, 116, 4391.
Liu Y., Snow B. E., Kickhoefer V. A., Erdmann N., Zhou W., Wakeham A., Gomez M., Rome L. H., H. L., Mol. Cell. Biol. 2004, 24, 5314.
K. J. Dillon, G. C. M. Smith, N. M. B. Martin, J. Biomol. Screen 2003, 8, 347.
P. Decker, E. A. Miranda, G. de Murcia, S. Muller, Clin. Cancer Res. 1999, 5, 1169.
K. S. Putt, P. J. Hergenrother, Analytical Biochemistry 326 (2004) 78-86; An enzymatic assay for poly(ADP-ribose) polymerase-1 (PARP-1) via the chemical quantitation of NAD⁺: application to the high-throughput screening of small molecules as potential inhibitors.
A. Cheung, J. Zhang, Anal. Biochem. 2000, 282, 24.
R. W. Johnson, T. M. Marschner, N. J. Oppenheimer, J. Am. Chem. Soc. 1988, 110, 2257.
S. Tono-oka, I. J. Azuma, J. Heterocyclic Chem. 1989, 26, 339.
S. Tono-oka, I. Azuma, Liebigs Ann./Recueil 1997, 1823.
S. Tono-oka, M. Hatakeyama, Chem. Pharm. Bull. 2001, 49, 123.
S. Yamada, Q. M. Gu, C. J. Sih, J. Am. Chem. Soc. 1994, 116, 10787.
K. Takahashi, A. A. Pieper, S. E. Croul, J. Zhang, S. H. Snyder, J. H. Greenberg, Brain Res. 1999, 829, 46.
M. J. Eliasson, K. Sampei, A. S. Mandir, P. D. Hurn, R. J. Traystman, J. Bao, A. Pieper,
Z. Q. Wang, T. M. Dawson, S. H. Snyder, V. L. Dawson, Nat. Med. 1997, 3, 1089.
F. Moroni, E. Meli, F. Peruginelli, A. Chiarugi, A. Cozzi, R. Picca, P. Romagnoli, R. Pellicciari, D. E. Pellegrini-Giampietro, Cell Death Differ. 2001, 8, 921.
Kawaici M et al., J. Biol. Chem. 1981, 256, 9483.
Beneke S et al., Exp. Gerontol. 2000, 35, 989.
Banasik M et al., Acta Neurobiol. Exp. 2004, 64, 4.
Rippmann J F et al., J. Mol. Biol. 2003, 325, 1107.
Holleman A et al., 2005, Decreased PARP and procaspase-2 protein levels are associated with cellular drug resistance in childhood acute lymphoblastic leukemia; Blood 106(5):1817-1823.
Biochem. Biophys. Acta, 950: 147-160, 1988.
FASEB Journal 10: 587-597, 1996.
Science, 267: 1456-1462, 1995.

1. A compound having the formula CX-1:

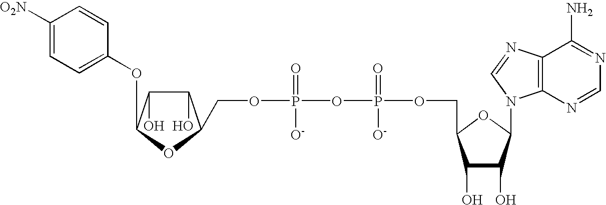

2. A method for detecting a poly(ADP-ribose) polymerase (PARP) enzyme activity, comprising providing a test sample putatively containing PARP enzymatic activity, reacting said sample with a calorimetric substrate, and observing a reaction product, thereby detecting said PARP enzyme activity.

3. The method of claim 2 wherein said calorimetric substrate is a derivative of nicotinamide adenine dinucleotide (NAD).

4. The method of claim 2 wherein said calorimetric substrate is formed from beta-NAD⁺ and para-nitrophenol.

5. The method of claim 2 wherein said colorimetric substrate is compound CX-1:

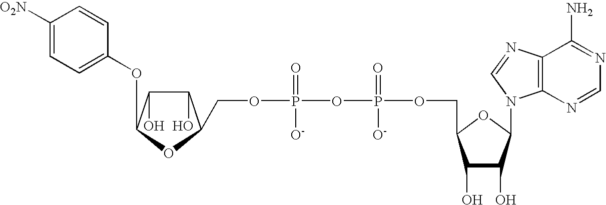

6. The method of claim 2 wherein said PARP enzyme activity is an activity of a PARP enzyme selected from the group consisting of PARP-1, tankyrase-1 (PARP-5), and VPARP (PARP-4).

7. The method of claim 2 further comprising kinetically monitoring said PARP enzyme activity, wherein said monitoring is achieved by performing a first observing step and at least a second observing of said reaction product, wherein said first and second observing steps are performed at different times.

8. The method of claim 2 further comprising kinetically monitoring said PARP enzyme activity, wherein said monitoring is achieved by providing at least a first test sample and a second test sample, independently reacting in separate reactions said samples with said colorimetric substrate, and independently observing said reaction products, wherein said observing occurs after different time periods of said reacting.

9. The method of claim 2 wherein said detecting further comprises providing a test substance in said test sample, wherein said test substance is a putative modifier of a PARP activity.

10. A method of screening for a substance putatively capable of modifying a PARP enzyme activity, comprising:
(a) providing a test material with putative PARP enzyme modification capability;
(b) providing a PARP enzyme;
(c) reacting in a test reaction said test material and said PARP enzyme with a PARP colorimetric substrate; and
(d) observing a reaction product of said reacting step;
thereby screening for said material capable of modifying a PARP enzyme.

11. The method of screening of claim 10 wherein said substance is putatively capable of inhibiting a PARP enzyme activity.

12. The method of screening of claim 10 wherein said substance is putatively capable of potentiating a PARP enzyme activity.

13. The method of claim 10 wherein said screening is high throughput screening and further comprises:
(e) providing at least a second test material; and
(f) in the first test reaction, independently in a second test reaction, or both in the first and second reactions; reacting said PARP enzyme with said PARP calorimetric substrate in the presence of said second test material; and
(g) if said second reaction is performed, observing a second reaction product.

14. A substrate compound capable of reacting specifically with a PARP enzyme, wherein said substrate is capable of forming a colorimetric product upon reaction with said PARP enzyme.

15. The substrate compound of claim 14 wherein said PARP enzyme is PARP-1, tankyrase-1 (PARP-5), or VPARP (PARP-4).

16. A method of synthesizing a substrate for a PARP enzyme, comprising providing a nicotinamide adenine dinucleotide component, providing a nitrophenol component, and reacting said components, thereby generating said PARP enzyme substrate.

17. The method of claim 14 wherein said substrate is a non-fluorescent substrate.

18. The method of claim 14 wherein said substrate is a calorimetric substrate.

19. A composition comprising compound CX-1.

20. A kit for detecting a presence, absence, or level of a PARP enzyme activity, comprising a PARP-specific colorimetric substrate and at least one control sample, wherein said one control sample is either a positive control sample capable of exhibiting PARP enzyme activity or a negative control sample which lacks PARP activity.

21. The kit of claim 20 further comprising a modifier compound, wherein said modifier compound is capable of inhibiting a PARP enzyme activity or potentiating a PARP enzyme activity.

22. The kit of claim 20 wherein said calorimetric substrate is formed from a nicotinamide adenine dinucleotide component and a nitrophenol component.

23. The kit of claim 20 wherein said calorimetric substrate is compound CX-1.

24. The kit of claim 20 wherein said PARP enzyme activity is an activity of a PARP enzyme selected from the group consisting of PARP-1, tankyrase-1 (PARP-5), and VPARP (PARP-4).

25. The kit of claim 20 further comprising a solvent for said calorimetric substrate.

26. The kit of claim 20 further comprising a second control sample, wherein said second control sample is a negative control sample if the first control sample is a positive control sample, and vice versa.
